(19) United States
(12) Patent Application Publication
(10) Pub. No.: US 2016/0278851 A1
(43) Pub. Date: Sep. 29, 2016

(54) STEERABLE CATHETER HAVING PULL WIRES AFFIXED WITHIN THE DISTAL PORTION OF THE CATHETER

(71) Applicant: Medtronic CryoCath LP, Toronto (CA)

(72) Inventors: Madeline A. MANNION, Beverly, MA (US); Gary R. FIEDLER, Forest Lake, MN (US); John B. HORRIGAN, Beverly, MA (US); Stuart R. MACDONALD, Andover, MA (US)

(21) Appl. No.: 14/665,499

(22) Filed: Mar. 23, 2015

(51) Int. Cl. A61B 18/14; A61B 5/00; A61M 25/00; A61M 25/01

(52) U.S. Cl. CPC A61B 18/1492; A61M 25/0147; A61B 5/6852; A61M 25/0012; A61M 25/0009; A61M 25/005; A61B 2018/00839

(57) ABSTRACT

A method and system with enhanced steerability and simplicity of construction for medical devices and a method of manufacturing the same. A steerable medical device may include an elongate body having a distal portion, proximal portion, and central lumen extending therethrough, a distal tip coupled to the distal portion of the elongate body, and at least one pull wire, each of the at least one pull wire having a distal end coupled to the distal tip, the distal portion of the elongate body including at least one pull wire lumen extending proximally from the distal tip through the distal portion of the elongate body, each of the at least one pull wire being within a corresponding pull wire lumen of the at least one pull wire lumen in the distal portion of the elongate body and within the central lumen in the proximal portion of the elongate body.

### CROSS-REFERENCE TO RELATED APPLICATION

n/a

### STATEMENT REGARDING FEDERALLY SPONSORED RESEARCH OR DEVELOPMENT

n/a

### FIELD OF THE INVENTION

The present invention relates to a method and system with enhanced steerability and simplicity of construction for medical devices and a method of manufacturing same.

### BACKGROUND OF THE INVENTION

Catheters are commonly used to perform medical procedures within very small spaces in a patient's body, and most procedures mandate precise catheter navigation. A catheter used to perform many ablation and mapping procedures generally includes a handle and a flexible elongate body or shaft having a distal end. Steering the distal end of a catheter can be difficult, especially as the elongate body passes through a tortuous vascular path.

Catheter tip steering is often accomplished with the use of one or more pull wires embedded within the wall of the elongate body. Typically, the one or more pull wires are embedded within the wall for the entire length of the elongate body or for most of the length of the elongate body, from the distal end to, or to a location proximate, the handle and/or steering mechanism of the device. In order to construct such a device, however, the wall of the elongate body must be manufactured in several smaller sections that are then bonded together. Not only does this greatly increase the complexity of construction of the device, but may also compromise the integrity of the elongate body because it is not manufactured as a single piece. Additionally, such construction requires that the elongate body wall and one or more pull wires are added to the device on the outside of a braid or mesh, which is typically included in the elongate body to add strength and resilience. As a result, the one or more pull wires may break through the elongate body wall if excess pull force is exerted on the pull wires during a procedure.

Additionally, devices in which the one or more pull wires are embedded in the elongate body wall for the entire or substantially the entire length of the elongate body may be difficult to steer. That is, the distal end of an elongate body of this construction may have a poor torque response and an exaggerated whipping effect during rotation. Bonding multiple wall segments together to create the elongate body also sacrifices torque of the elongate body.

It is therefore desired to provide a medical system, device, and method of construction thereof that is simple to manufacture, reduces the risk of elongate body wall breakage, and improves torque response and steerability.

### SUMMARY OF THE INVENTION

The present invention advantageously provides a device and system with enhanced steerability and simplicity of construction for medical devices and a method of manufacturing the same. A steerable medical device may include an elongate body having a distal portion, a proximal portion, and a central lumen extending therethrough, a distal tip coupled to the distal portion of the elongate body, and at least one pull wire, each of the at least one pull wire having a distal end coupled to the distal tip, the distal portion of the elongate body including at least one pull wire lumen (for example, two pull wire lumens) extending proximally from the distal tip through the distal portion of the elongate body, each of the at least one pull wire being within a corresponding pull wire lumen of the at least one pull wire lumen in the distal portion of the elongate body and with the central lumen in the proximal portion of the elongate body. The elongate body may include a first layer of material, the central lumen and at least one pull wire lumen being defined by the first layer of material. The elongate body may further include a second layer of material that at least substantially surrounds the first layer of material, the second layer of material being braided. The second layer may surround the first layer of material on the proximal portion and the distal portion of the elongate body. The device may further include a handle coupled to the proximal portion of the elongate body, the second layer extending from a location proximate the handle to a location proximate the distal tip. The elongate body may further include a third layer of material that at least substantially surrounds the second layer of mater. The third layer may be a single continuous piece of material. The distal tip may include an electrode, and in one embodiment the electrode may include a plurality of orifices. The distal portion of elongate body may have a first length and the proximal portion of the elongate body may have a second length, the second length being greater than the first length.

A medical device may include an elongate body having a distal end, a distal portion, a proximal end, a proximal portion, a central lumen extending between the distal end and the proximal end, and at least one pull wire lumen within the elongate body distal portion, and a distal tip coupled to the elongate body distal portion, the elongate body including an inner first layer, a braided second layer, and an outer third layer, each of the inner first layer, the braided second layer, and the outer third layer extending between the elongate body proximal end and the elongate body distal end.

A method of manufacturing a steerable medical device may include: surrounding each of at least one minor mandrel with a first sheath; inserting each of the at least one minor mandrel into one of an at least one groove provided on a major mandrel having a first portion and a second portion, each of the at least one groove being located on an outer surface of the major mandrel second portion; surrounding the major mandrel and at least one minor mandrel with a second sheath; overlaying a braided layer over the second sheath; overlaying a third sheath over the braided layer; removing the major mandrel to create an elongate body having a first portion, a second portion, and a central lumen extending therebetween; removing the at least one minor mandrel to create at least one pull wire lumen within the elongate body second portion; and attaching a distal tip to the elongate body second portion. The first sheath, the second sheath, and the third sheath may be composed of a polymer, such as PTFE. The braided layer may be formed from metal filament, such as stainless steel filaments. A distal end of at least one pull wire may be affixed to the distal tip, at least a portion of the at least one pull wire being unaffixed to the distal tip. The method may also include passing the unaffixed portion of each of the at least one pull wire through a corresponding one of the at least one pull wire lumen and then into the central lumen. Each of the at least one pull wire may be located within the corresponding one of the at least one pull wire lumen in the elongate body second portion and located within the central lumen in the elongate body first portion.

### BRIEF DESCRIPTION OF THE DRAWINGS

A more complete understanding of the present invention, and the attendant advantages and features thereof, will be more readily understood by reference to the following detailed description when considered in conjunction with the accompanying drawings wherein:

FIG. 1 shows an exemplary system including a medical device with enhanced steerability;

FIG. 2 shows a cross-sectional view of a distal portion of a medical device having an elongate body with enhanced steerability;

FIG. 3 shows a cross-sectional view of the elongate body along line 3-3 shown in FIG. 1;

FIG. 4 shows a cross-sectional view of the elongate body along line 4-4 shown in FIG. 1; and

FIGS. 5-10 show steps in a method of manufacturing an elongate body with enhanced steerability.

### DETAILED DESCRIPTION OF THE INVENTION

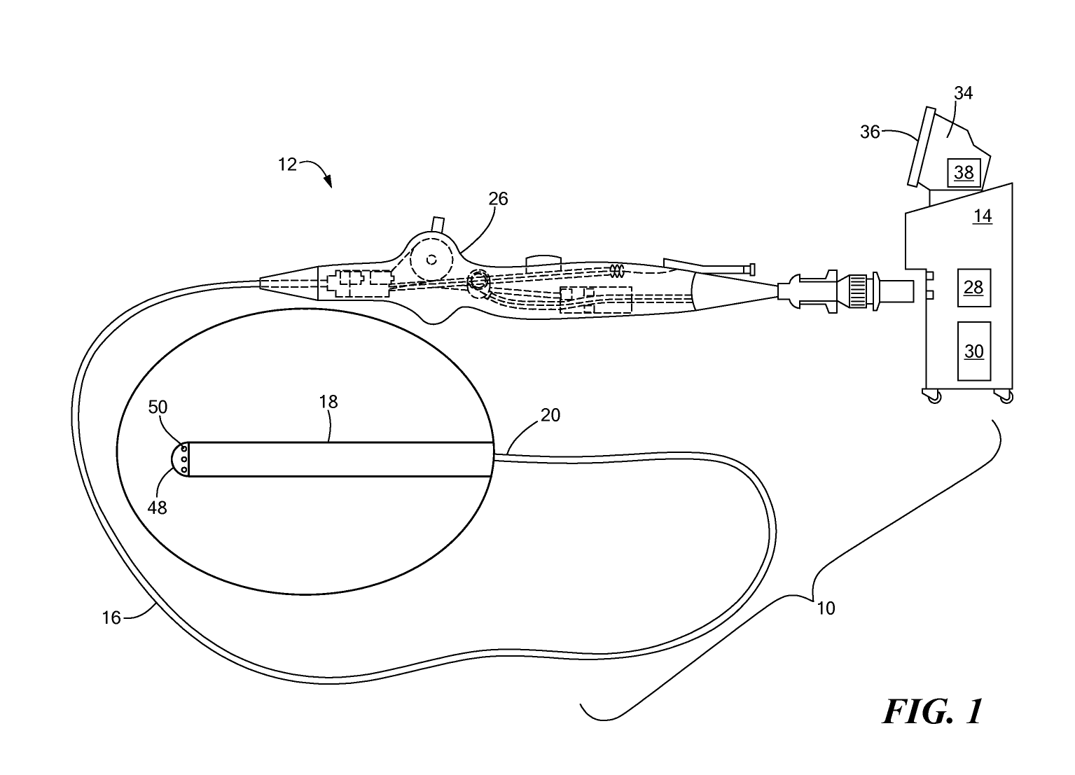

Referring now to FIGS. 1-4, an exemplary system and medical device having enhanced steerability are shown. As shown in FIG. 1, an exemplary system 10 is shown. The system 10 may generally include a medical device 12 (for example, an ablation or mapping catheter) and a console 14. The medical device 12 may include an elongate body 16 having a distal portion 18, a proximal portion 20, and one or more lumens therein. For example, the elongate body 16 may include or define a central lumen 22. The distal portion 18 of the elongate body 16 may be capable of in-plane and/or out-of-plane deflection and is steerable by one or more pull wires 24. The pull wires 24 may be composed of stainless steel, nitinol, titanium, alloy, polymer, or other suitable material. The proximal portion 20 of the elongate body 16 may be affixed to a handle 26 having various inlets, outlets, steering control mechanisms (for example, knobs, toggles, etc.). Further, the one or more pull wires 24 may be either coupled to or routed through the handle 26.

The medical device 12 may be in fluid and/or electrical communication with the console 14, which may be adapted for use with one or more energy modalities and/or medical procedure that is facilitated by a steerable catheter, including cardiac mapping. For example, the console 14 may include an energy generator 28 (for example, a radiofrequency generator), and/or a refrigerant reservoir or other fluid reservoir 30. For example, the device 12 may be in fluid communication with a saline reservoir if the device 12 is an irrigated catheter. Additionally or alternatively, the console 14 may also include a laser, microwave, and/or ultrasound energy source. The console 14 may also include a computer 34, a display 36, and/or various user control devices (for example, buttons, knobs, valves, keyboard, touch screen, foot pedals, etc.). The computer 34 may include one or more processors 38 programmable to execute one or more algorithms for receiving and processing data received from the device 12. As used herein, the term “console” includes any system components that are not part of the medical device itself, whether or not a particular component is physically located within or external to the console 14.

Although the system 10 is shown as including a focal catheter, it will be understood that the system 10 may additionally or alternatively include any other medical device used for a treatment, mapping, or other medical procedure, including a balloon catheter, a catheter having an expandable electrode array, a clamp with multiple jaws, an electrosurgical device, or the like.

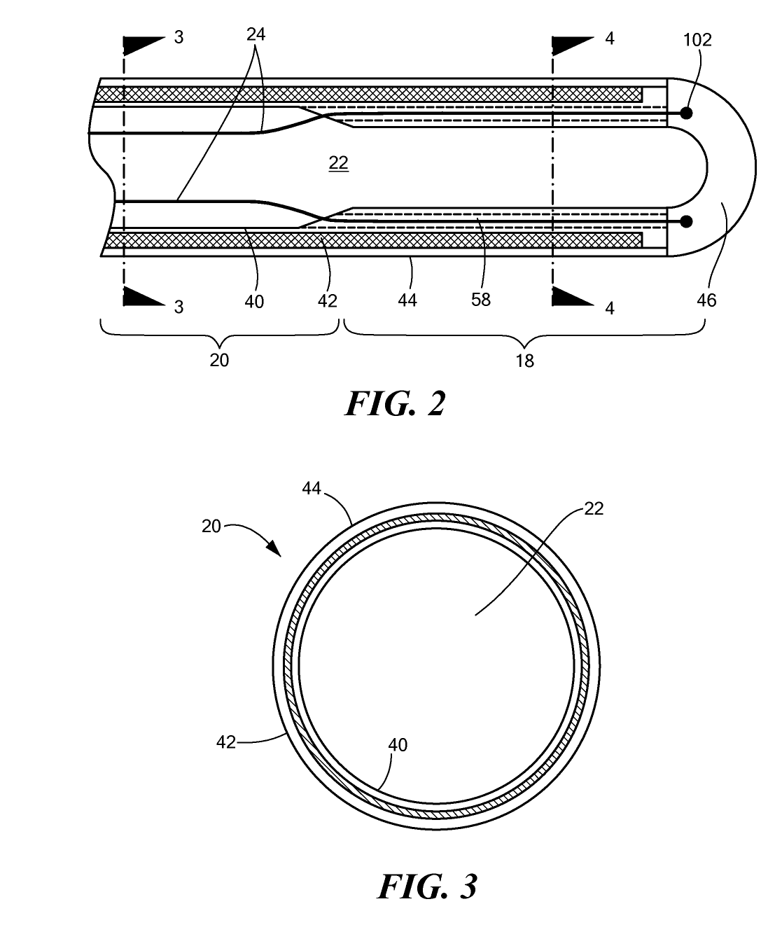

As shown in more detail in FIGS. 2-4, the elongate body 16 may include at least three layers: an inner first layer 40, a braided second layer 42, and an outer third layer 44. The inner first layer 40 and outer third layer 44 may each be composed of a biocompatible polymer material, such as but not limited to polytetrafluoroethylene (PTFE), high density polyethylene, PEBAX®, and/or polyurethane. The layers 40, 42, 44, may be coaxial with each other. The braided second layer 42 may be composed of a braided or meshed material, such as stainless steel, Nitinol, or other metal. The inner first layer 40 may define or surround one or more lumens, such as the central lumen 22 of the elongate body 16, and may extend from the proximal end to the distal end of the elongate body. The braided second layer 42 may be located between the inner first layer 40 and the outer third layer 44, and may extend from the proximal end to the distal end of the elongate body, or to a location proximate the distal end of the elongate body. The outer third layer 44 may be overmolded on the braided second layer 42 during the elongate body 16 manufacturing process, and may extend from the proximal end to the distal end of the elongate body. The inner first layer 40 and the outer third layer 44 each may be composed of a single continuous piece of material, rather than of multiple segments of material that are bonded or coupled together. Further, the braided second layer 42 may extend over the entire length of the elongate body 16 or from the proximal end of the elongate body 16 to a location proximate a distal tip 46.

The distal portion 18 may include the distal tip 46. Depending on the type of procedure for which the device is used, the distal tip 46 may be conductive or nonconductive. In the exemplary embodiment shown in FIGS. 1 and 2, the distal tip 46 may be defined by a thermally conductive element, such as an electrode 48. Alternatively, if the device 12 is to be used with laser energy, the distal tip 46 may be defined by a translucent or transparent material, such as glass. Still further, the distal tip 46 may include one or more orifices or apertures 50 (for example, as shown in the non-limiting embodiment in FIG. 1) for the delivery of a fluid such as saline, as may be the case when the device 12 is an irrigated catheter. As shown in FIG. 2, the distal tip 46 may extend distally beyond the distal edge of the braided second layer 42. In some embodiments, the distal tip 46 may be at least substantially contiguous and continuous with the distal portion 18 of the elongate body 16 when coupled to the elongate body 16.

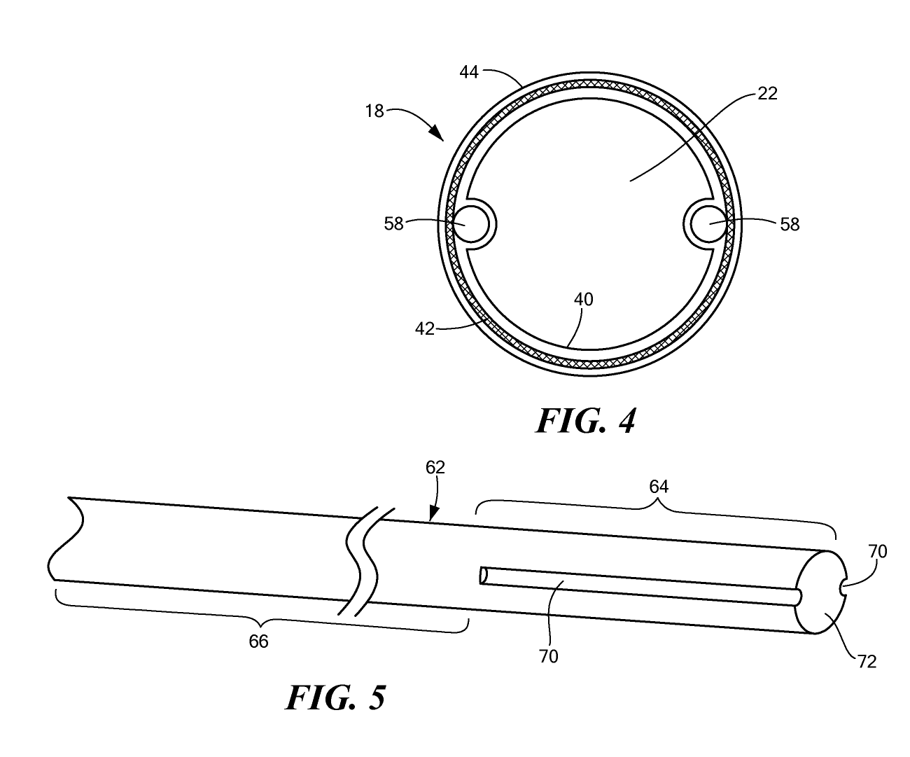

As shown in FIG. 2, the one or more pull wires 24 may be embedded or disposed within the inner first layer 40 in the elongate body distal portion 18, but may be located within the central lumen 22 throughout the elongate body proximal portion 20. For example, the distal portion 18 may include one or more pull wires lumens 58 that are in communication with the central lumen 22, and each pull wire 24 may be located within a pull wire lumen 58 in the distal portion 18 and within the central lumen 22 in the proximal portion 20. An exemplary cross-sectional view of the elongate body proximal portion 20 is shown in FIG. 3 and an exemplary cross-sectional view of the elongate body distal portion 18 is shown in FIG. 4. The pull wires lumens 58 may be disposed in a radially symmetrical configuration about the central lumen 22, or they may be asymmetrically disposed about the central lumen 22. The elongate body distal portion 18 may be of any length, but generally may be of a shorter length than the elongate body proximal portion 20. This configuration in which the pull wires are embedded only within the distal section of the catheter may improve torque response while still allowing for distal deflection. It may also eliminate the need for bonding multiple segments together, which can sacrifice torque and yield loss when dealing with braid termination in the bonding section. Although two pull wire lumens 58 are shown in FIG. 4, it will be understood that the elongate body 16 may include any number and configuration of pull wire lumens 58.

Referring now to FIGS. 5-10, a method of manufacturing an elongate body with enhanced steerability is shown. In the first step of the method (shown in FIG. 5), a central mandrel 62 is used that includes a first portion 64, which may correspond to the elongate body distal portion 18, and a second portion 66, which may correspond to the elongate body proximal portion 20. The second portion 66 of the central mandrel 62 may include one or more lateral grooves 70 that each extends a distance along the outer surface of the central mandrel 62 from a first end 72 at the first portion 64. The location at which the one or more grooves 70 end in the first portion 64 may mark the point between the first portion and the second portion 66 of the central mandrel 62.

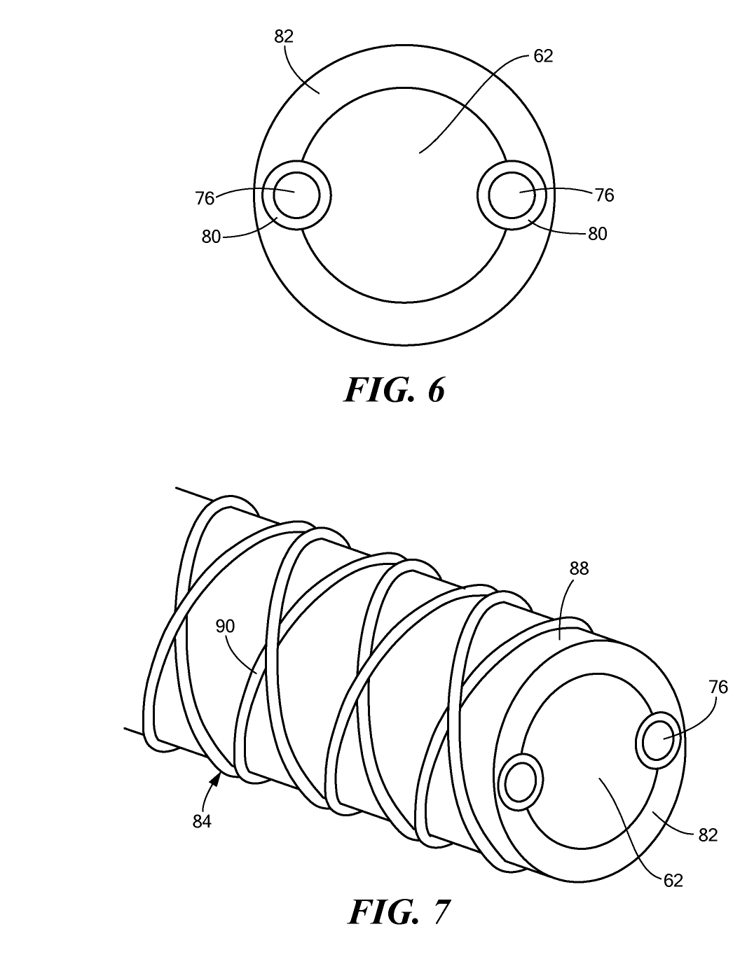

In the second step of the method (shown in FIG. 6), a smaller pull wire lumen mandrel 76 may be inserted into each of the lateral grooves 70 in the central mandrel 62. Further, each pull wire lumen mandrel 76 may itself be surrounded by an etched liner 80 (which may also be referred to as a jacket or sheath) composed of a biocompatible material, such as PTFE. The central mandrel 62 and pull wire lumen mandrels 76 together may be surrounded by an outer liner 82 (which may also be referred to as a jacket or sheath) of biocompatible material, such as PTFE. This liner 82 may become the inner first layer 40 of the elongate body 16. In a non-limiting configuration, the outer liner 82 may be thicker than the etched liner 80 surrounding each pull wire lumen mandrel 76 (for example, as shown in FIG. 6).

In the third step of the method (as shown in FIG. 7), a braid 84 is created on the outside of the outer liner 82, from a first end of the liner 82, which may become the proximal end of the elongate body 16, to a location at or proximate the second end 88 of the liner, which may become the distal end 18 of the elongate body 16. For example, the braid 84 may end a distance from the second end 88 of the liner, such as at a distance of approximately 2 mm from the second end 88. Alternatively, the braid 84 may continue to the second end 88 of the liner. This braid 84 may become the braided second layer 42 of the elongate body 16. The braid 84 may be composed of filaments, wires, or threads 90 of one or more biocompatible materials, such as metals or metal alloys (for example, stainless steel). The braid may be terminated at the second end 88 of the liner 80 by a standard technique, such as with the use of adhesives, glue, bonding agents, heat shrinking, or the like.

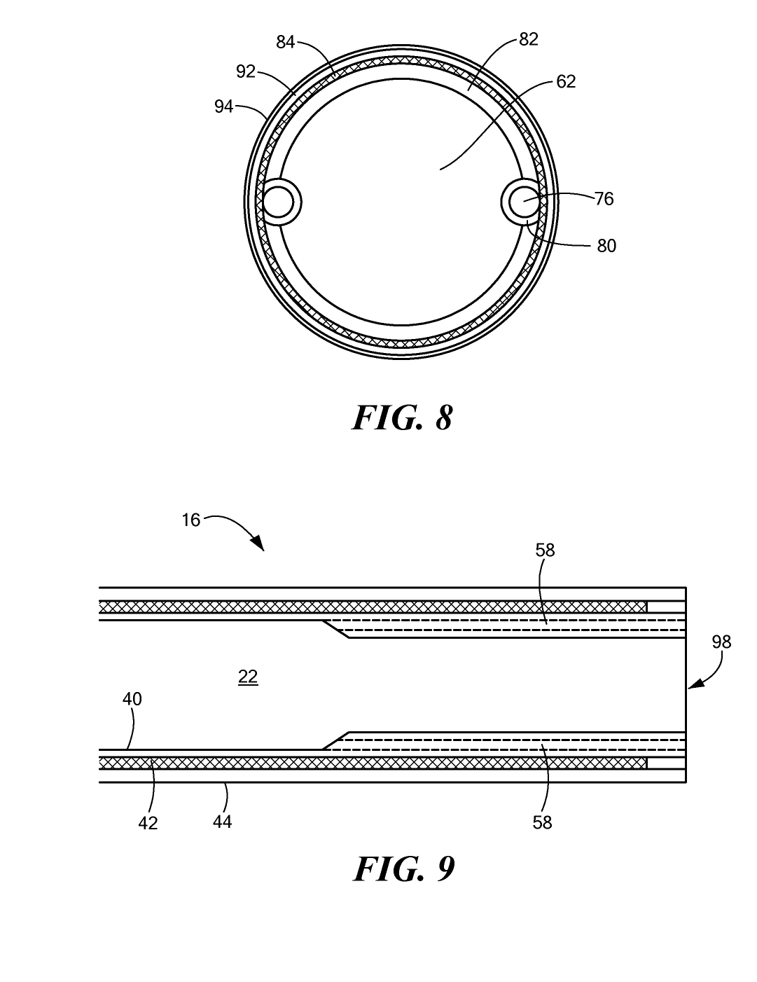

In the fourth step of the method (as shown in FIG. 8), an outer layer 92 of biocompatible polymer, such as PTFE, may be laid over the braid 84. A layer of heat shrink material 94 may be laid over the outer layer 92 and then reflowed together. The outer layer 92 of biocompatible material may become the outer third layer 44 of the elongate body 16.

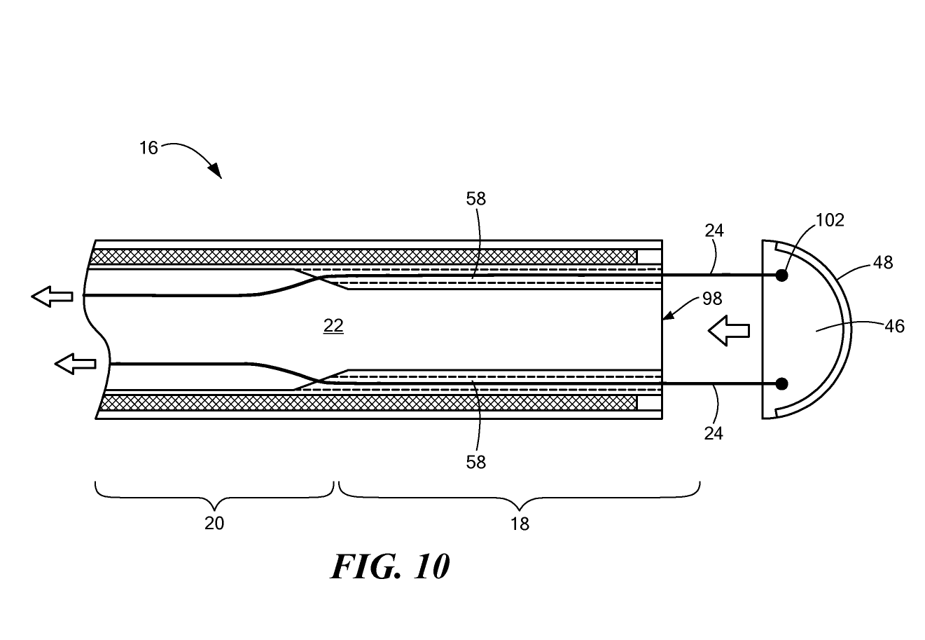

In the fifth step of the method (as shown in FIG. 9), the layer of heat shrink material 94 and the mandrels 62, 76 may be removed. At this stage, the resulting braided tubular composite structure may be referred to as the elongate body. The elongate body 16 may now include a central lumen 22 formed by the central mandrel 62 and one or more pull wire lumens 58 formed from the one or more pull wire lumen mandrels 76. In the sixth step of the method (as shown in FIG. 10), the distal tip 46 may be coupled to the elongate body distal end 98. The distal portions 100 of each of the one or more pull wires 24 may be coupled to the distal tip 46 by a standard technique at a connection point 102, such as by welding, adhering, or chemical bonding. When the distal tip 46 is placed in contact with the elongate body distal end 98, the pull wire lumens 58 may be described to extend proximally from the distal tip 46 through the distal portion 18 of the elongate body 16 to the point at which the proximal portion 20 of the elongate body is deemed to begin. The free portion of each pull wire 24 may be threaded through a corresponding pull wire lumen 58 in the distal portion 18 of the elongate body 16 and then into the central lumen 22 within the proximal portion 20 of the elongate body 16. Once the distal tip 46 and the elongate body distal end 98 are brought together, they may be joined by a standard technique, such as reflowing, chemical or heat bonding, adhesives, or the like. Additionally or alternatively, the distal tip 46 may be mechanically coupled to the distal portion 18, such as by a friction fit or by one or more matable tabs and grooves, ridges, or other structural features. For example, the distal portion 18 may include an insulative liner that is heat fused (reflowed) into the distal portion 18 of the elongate body 16, and the distal tip 46 may be mechanically coupled to the insulative liner, such as by a “snap-fit” connection, although adhesives may additionally be used. The proximal ends of the one or more pull wires 24 may be mechanically coupled to the handle 26 of the device or other actuation or steering mechanism.

It will be appreciated by persons skilled in the art that the present invention is not limited to what has been particularly shown and described herein above. In addition, unless mention was made above to the contrary, it should be noted that all of the accompanying drawings are not to scale. A variety of modifications and variations are possible in light of the above teachings without departing from the scope and spirit of the invention, which is limited only by the following claims.

What is claimed is:

1. A steerable medical device, comprising:
an elongate body having a distal portion, a proximal portion, and a central lumen extending therethrough;
a distal tip coupled to the distal portion of the elongate body; and
at least one pull wire, each of the at least one pull wire having a distal end coupled to the distal tip;
the distal portion of the elongate body including at least one pull wire lumen extending proximally from the distal tip through the distal portion of the elongate body, each of the at least one pull wire being within a corresponding pull wire lumen of the at least one pull wire lumen in the distal portion of the elongate body and within the central lumen in the proximal portion of the elongate body.

2. The steerable medical device of claim 1, wherein the elongate body includes a first layer of material, the central lumen and at least one pull wire lumen being defined by the first layer of material.

3. The steerable medical device of claim 2, wherein the elongate body further includes a second layer of material that at least substantially surrounds the first layer of material, the second layer of material being braided.

4. The steerable medical device of claim 3, wherein the braided second layer is continuous and extends between the proximal portion of the elongate body and the distal tip.

5. The steerable medical device of claim 3, wherein the second layer surrounds the first layer of material on the proximal portion and the distal portion of the elongate body.

6. The steerable medical device of claim 5, further comprising a handle coupled to the proximal portion of the elongate body, the second layer extending from a location proximate the handle to a location proximal to and proximate the distal tip.

7. The steerable medical device of claim 3, wherein the elongate body further includes a third layer of material that at least substantially surrounds the second layer of material.

8. The steerable medical device of claim 7, wherein the third layer of material is a single continuous piece of material.

9. The steerable medical device of claim 5, wherein the distal tip includes an electrode.

10. The steerable medical device of claim 9, wherein the electrode includes a plurality of orifices.

11. The steerable medical device of claim 1, wherein distal portion of the elongate body includes two pull wire lumens.

12. The steerable medical device of claim 1, wherein the distal portion of elongate body has a first length and the proximal portion of the elongate body has a second length, the second length being greater than the first length.

13. A medical device, comprising:
an elongate body having a distal end, a distal portion, a proximal end, a proximal portion, a central lumen extending between the distal end and the proximal end, and at least one pull wire lumen within the elongate body distal portion; and
a distal tip coupled to the elongate body distal portion,
the elongate body including an inner first layer, a braided second layer, and an outer third layer, each of the inner first layer, the braided second layer, and the outer third layer extending between the elongate body proximal end and the elongate body distal end.

14. A method of manufacturing a steerable medical device, the method comprising:
surrounding each of at least one minor mandrel with a first sheath;
inserting each of the at least one minor mandrel into one of an at least one groove provided on a major mandrel having a first portion and a second portion, each of the at least one groove being located on an outer surface of the major mandrel second portion;
surrounding the major mandrel and at least one minor mandrel with a second sheath;
overlaying a braided layer over the second sheath;
overlaying a third sheath over the braided layer;
removing the major mandrel to create an elongate body having a first portion, a second portion, and a central lumen extending therebetween;
removing the at least one minor mandrel to create at least one pull wire lumen within the elongate body second portion; and
attaching a distal tip to the elongate body second portion.

15. The method of claim 14, wherein the first sheath, the second sheath, and the third sheath are composed of a polymer.

16. The method of claim 15, wherein the polymer is PTFE.

17. The method of claim 14, wherein the braided layer is formed from metal filaments.

18. The method of claim 17, wherein the metal filaments are composed of stainless steel.

19. The method of claim 14, wherein a distal end of at least one pull wire is affixed to the distal tip, at least a portion of the at least one pull wire being unaffixed to the distal tip.

20. The method of claim 19, further comprising:
passing the unaffixed portion of each of the at least one pull wire through a corresponding one of the at least one pull wire lumen and then into the central lumen.

21. The method of claim 20, wherein each of the at least one pull wire is located within the corresponding one of the at least one pull wire lumen in the elongate body second portion and located within the central lumen in the elongate body first portion.
